(19) United States
(12) Patent Application Publication
(10) Pub. No.: US 2019/0328221 A1
(43) Pub. Date: Oct. 31, 2019

(54) Wireless Optical Curette System

(71) Applicant: The Regents of the University of California, Oakland, CA (US)

(72) Inventors: Cherie Yvette Wink, Yorba Linda, CA (US); Petra Beate Wilder Smith, San Juan Capistrano, CA (US)

(73) Assignee: The Regents of the University of California, Oakland, CA (US)

(21) Appl. No.: 16/343,287

(22) PCT Filed: Oct. 20, 2017
(86) PCT No.: PCT/US2017/057713

§ 371 Date: Apr. 18, 2019

Related U.S. Application Data
(60) Provisional application No. 62/413,423, filed on Oct. 26, 2016.

(51) Int. Cl. A61B 1/24; A61B 1/07; A61B 1/04; A61B 1/00; A61C 3/00; A61B 1/06

(52) U.S. Cl. CPC A61B 1/24; A61B 1/07; A61B 1/042; A61B 1/00016; A61B 1/00032; A61B 1/0669; A61B 1/0638; A61B 1/00006; A61C 3/00

(57) ABSTRACT

A wireless optical curette system is disclosed for allowing a user to visually examine in real-time and debride a target surface within a cavity of a patient. In at least one embodiment, at least one instrument attachment provides at least one fiber optic bundle extending between a working end of the instrument attachment and an opposing engagement end of the instrument attachment. An imaging assembly is selectively engagable with the engagement end of the instrument attachment and provides at least one light source and at least one imaging sensor in selective optical communication with the at least one fiber optic bundle, along with at least one microprocessor configured for wirelessly transmitting captured digital images, via at least one transceiver positioned within the imaging assembly, to at least one of a computing device and an imaging display for displaying the digital images in real-time.

### RELATED APPLICATIONS

This application claims priority and is entitled to the filing date of U.S. provisional application Ser. No. 62/413,423, filed on Oct. 26, 2016. The contents of the aforementioned application are incorporated herein by reference.

### BACKGROUND

The subject of this patent application relates generally to dental instruments, and more particularly to a wireless optical curette system configured for allowing a user of the system to visually examine in real-time and debride a cavity of a patient.

Applicant(s) hereby incorporate herein by reference any and all patents and published patent applications cited or referred to in this application.

By way of background, in the field of dentistry, gingival and periodontal disease affect a large number of the population. This disease encompasses a group of disorders affecting soft tissue attachment and bone support around the teeth. It is often desirable to examine the teeth and tissues subgingivally in order to detect the presence of various conditions and diseases including anatomical features and manifestations of gingival and/or periodontal or other diseases. Such conditions and diseases may include enamel, dentinal or cemental surface irregularities such as scaling grooves and cemental tears, root fractures, defective restorative margins and the presence of plaque biofilm and calculus. Plaque biofilm is associated with the development of periodontal disease and typically begins with formation on the interproximal and cervical third surfaces of teeth. Subgingival biofilm results from the apical proliferation of microorganisms from supragingival biofilm. Microorganisms make up at least 70%-80% of the solid matter which is higher in subgingival biofilm than in supragingival. Mineralization of the biofilm can begin as early as 24 to 48 hours when a patient's personal oral hygiene is neglected resulting in calculi. Calculus results from the deposition of minerals, such as calcium and phosphorus, into this plaque biofilm organic matrix giving rise to the hardened calculi on the supragingival and subgingival surfaces of the tooth. Bacteria held within the rough surface of the calculus perpetuate the inflammatory process leading to destruction of the connective tissue and bone.

An essential component of successful therapy requires complete removal of calculus and plaque biofilm from the tooth root surface to arrest the disease process and achieve oral health. Effective instrumentation techniques rely upon tactile and auditory feedback as well as working blindly subgingivally with a goal of achieving glass-like root surfaces to ensure that all attached and embedded deposits have been removed. Because there currently exists no mechanism for direct visualization of the tooth/root/periodontal structures, there exists no definitive endpoint for such techniques, which may lead to either over-instrumentation and unnecessary removal of tooth cementum or incomplete removal of the etiological factors. These traditional techniques offer no information on tooth/root/periodontal structure microanatomy, the persistence of localized areas of residual deposits or guidance on the appropriate end-point for non-surgical periodontal therapy or prophylactic procedures. Therefore, complete and optimal removal of all deposits in periodontal pockets is impossible with traditional scaling and root planning (“SRP”).

Aspects of the present invention fulfill these needs and provide further related advantages as described in the following summary.

### SUMMARY

Aspects of the present invention teach certain benefits in construction and use which give rise to the exemplary advantages described below.

The present invention solves the problems described above by providing a wireless optical curette system for allowing a user to visually examine in real-time and debride a target surface within a cavity of a patient. In at least one embodiment, a wireless optical curette device provides an at least one instrument attachment providing an at least one fiber optic bundle extending between a working end of the instrument attachment and an opposing engagement end of the instrument attachment. An imaging assembly has a first end selectively engagable with the engagement end of the at least one instrument attachment. The imaging assembly provides an at least one light source in selective optical communication with the at least one fiber optic bundle and configured for delivering an amount of controlled light to the target surface via the at least one fiber optic bundle. An at least one imaging sensor is positioned within the imaging assembly in selective optical communication with the at least one fiber optic bundle and configured for receiving an amount of controlled light, via the at least one fiber optic bundle, reflected from the target surface, and converting the reflected light into an at least one digital image of the target surface. An at least one microprocessor is positioned within the imaging assembly and configured for wirelessly transmitting the at least one digital image, via an at least one transceiver positioned within the imaging assembly, to at least one of an at least one computing device and an at least one imaging display for displaying the at least one digital image in real-time. The engagement end of the at least one instrument attachment and the first end of the imaging assembly cooperate to provide an optical port therebetween, the optical port configured for placing the at least one fiber optic bundle of the instrument attachment in optical communication with each of the at least one imaging sensor and at least one light source, when the instrument attachment is selectively engaged with the imaging assembly. Thus, the imaging assembly is capable of being removably engaged quickly and easily with a wide variety of different instrument attachments.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a simplified schematic view of an exemplary wireless optical curette system, in accordance with at least one embodiment;

FIGS. 2 and 3 are partial perspective views of an exemplary wireless optical curette device, in accordance with at least one embodiment;

FIG. 4 is an internal diagram of an exemplary imaging assembly of the exemplary curette device, in accordance with at least one embodiment;

FIG. 5 is a partial internal diagram of the exemplary imaging assembly and an exemplary instrument attachment of the exemplary curette device, in accordance with at least one embodiment; and

FIGS. 6 and 7 are perspective views of further exemplary curette devices, in accordance with at least one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

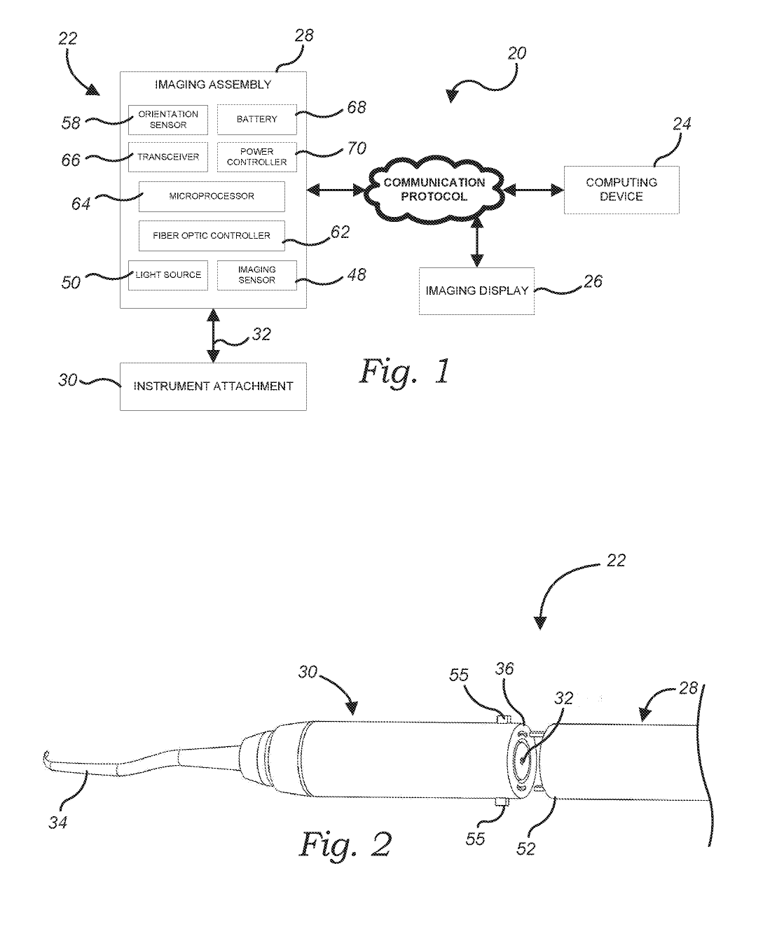

Turning now to FIG. 1, there is shown a simplified schematic view of an exemplary wireless optical curette system 20 configured for allowing a user of the system 20 (such as a doctor or clinician, for example) to view, examine, aid in diagnosis, and debride an oral cavity of a patient, including the teeth and periodontal tissues of the patient. It should be noted that while the system 20 is discussed primarily in the context of oral cavities for illustrative purposes, in further embodiments, the system 20 may be utilized in any other context—now known or later developed—where a curette or similar instrument might be utilized, such as removing impacted ear wax, curettage of the uterus, excision of adenoids, etc. Accordingly, the present invention should not be read as being limited to the dental context. In at least one embodiment, the system 20 includes a wireless optical curette device 22 configured for capturing and transmitting images of the patient's oral cavity in real-time, as discussed further below. In at least one embodiment, the curette device 22 is in selective communication with at least one of an at least one computing device 24 and an at least one imaging display 26, the at least one computing device 24 and imaging display 26 configured for receiving and displaying the images transmitted by the curette device 22. In at least one embodiment, the at least one computing device 24 is further configured for storing the images transmitted by the curette device 22.

It should be noted that communication between each of the curette device 22, at least one computing device 24, and at least one imaging display 26 may be achieved using any wired- or wireless-based communication protocol (or combination of protocols) now known or later developed. As such, the present invention should not be read as being limited to any one particular type of communication protocol, even though certain exemplary protocols may be mentioned herein for illustrative purposes. It should also be noted that the term “computing device” is intended to include any type of computing or electronic device now known or later developed having a display screen (or at least in communication with a display screen), such as desktop computers, mobile phones, smartphones, laptop computers, tablet computers, personal data assistants, gaming devices, wearable devices, etc. As such, the present invention should not be read as being limited to use with any one particular type of computing device, even though certain exemplary devices may be mentioned or shown herein for illustrative purposes. Additionally, the term “imaging display” is intended to include any type of standalone display device now known or later developed, such as a television, a display screen, heads-up-display enabled glasses or goggles, etc. As such, the present invention should not be read as being limited to use with any one particular type of imaging display, even though certain exemplary devices may be mentioned or shown herein for illustrative purposes.

In at least one embodiment, the curette device 22 incorporates components to allow for both illumination and magnification of a target area within the patient's oral cavity via a tetherless imaging assembly 28 that is selectively engagable with a variety of curettes and similar instruments (herein collectively referred to as “instrument attachments” 30). It is noted that, in at least one embodiment, each of the instrument attachment 30 and imaging assembly 28 is made of materials and shaped to support favorable ergonomics for the user by being relatively lightweight and allowing for a comfortable modified pen grasp during use, with precise finger placement in the modified pen grasp being critical to successful instrumentation. However, in further embodiments, each of the instrument attachment 30 and imaging assembly 28 may take on any other size, shape or dimensions, or may be constructed out of any material (or combination of materials), now known or later developed. As such, the particular embodiments illustrated in the accompany drawings are merely exemplary.

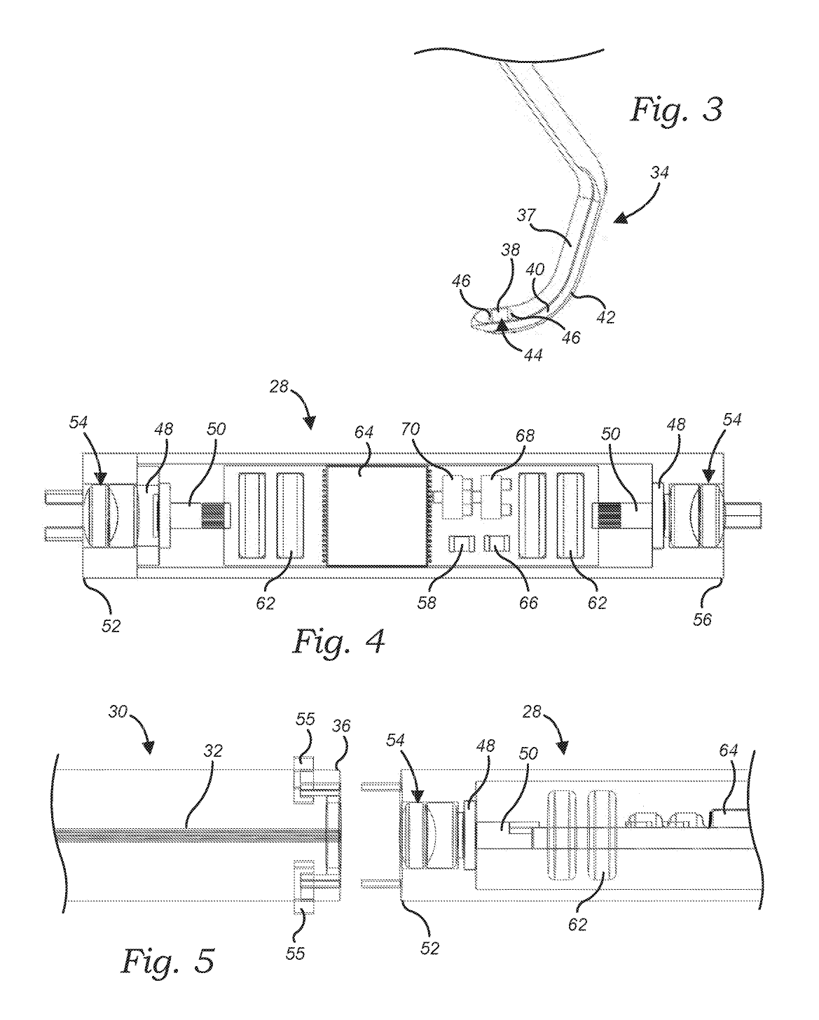

In at least one such embodiment, as illustrated in FIG. 2, the instrument attachment 30 provides an at least one fiber optic bundle 32 extending between a working end 34 of the instrument attachment 30 and an opposing engagement end 36 of the instrument attachment 30. In at least one embodiment, as best illustrated in FIG. 3, at least a portion of the fiber optic bundle 32 is housed in a separate encasement 37. At the engagement end 36 of the instrument attachment 30, the fiber optic bundle 32 is configured for being selectively connected with supporting components positioned within the imaging assembly 28, when the instrument attachment 30 is selectively engaged with the imaging assembly 28, as discussed further below. Additionally, in at least one embodiment, as best illustrated in FIG. 3, the working end 34 of the instrument attachment 30 provides a lens 38 in optical communication with the fiber optic bundle 32, the lens 38 providing a field of view showing the target area within the patient's oral cavity relative to the position of the working end 34 of the instrument attachment 30. In at least one embodiment, the field of view is in substantially the same plane as a face 40 of the working end 34 of the instrument attachment 30—i.e., the surface of the working end 34 looking substantially normal to a surface to be scraped (i.e., the target surface) by the working end 34, such as a tooth of the patient for example, during use of the curette device 22. In at least one embodiment, a focal length of the lens 38 is set to allow for the lens 38 to sit close to a cutting edge 42 of the working end 34 with minimal impact on the overall width of the working end 34 which, in turn, facilitates retraction of the soft tissue of the patient, maximizing the field of view and reducing tissue trauma. In at least one embodiment, the lens 38 is simply a trimmed and polished terminal end 44 of the fiber optic bundle 32. In at least one embodiment, the at least one fiber optic bundle 32 includes multiple fibers: an at least one light fiber to supply a controlled light 46, and an at least one return fiber which receives the reflected light from the target tissue surface. This reflected light captured by the return fiber is transmitted along the return fiber to an imaging sensor 48 positioned within the imaging assembly 28. In at least one embodiment, an at least one light source 50 is also positioned within the imaging assembly 28 for being in selective optical communication with the at least one light fiber when the instrument attachment 30 is selectively engaged with the imaging assembly 28, thereby providing the controlled light via the at least one light fiber. In at least one such embodiment, the at least one light source 50 is an at least one LED. In at least one alternate embodiment, the at least one light source 50 is an external source, such as an at least one mirror or an at least one LED mounted on the instrument attachment 30 in the same general location as the lens 38 of the fiber optic bundle 32. In still further embodiments, any other type of light source 50, now known or later developed, may be substituted. In at least one embodiment, the at least one light source 50 is filtered to highlight dental plaque, tartar, calcium or other buildup. In at least one embodiment, the imaging assembly 28 incorporates a polarization filter (not shown) to reduce specular reflection. In at least one embodiment, the at least one light source 50 consists of two or more different colors of light for improving image contrast. In at least one further embodiment, the images captured by the curette device 22 are digitally enhanced to highlight dental plaque, tartar, calcium or other buildup.

In at least one embodiment, as best illustrated in FIGS. 4 and 5, the engagement end 36 of the instrument attachment 30 and a first end 52 of the imaging assembly 28 cooperate to provide an optical port 54 therebetween, the optical port 54 configured for placing the at least one fiber optic bundle 32 of the instrument attachment 30 in optical communication with each of the imaging sensor 48 and at least one light source 50, when the instrument attachment 30 is removably engaged with the imaging assembly 28. In at least one embodiment, the optical port 54 is an at least one lens. In at least one embodiment, as illustrated in FIG. 5, the first end 52 of the imaging assembly 28 slots into the engagement end 36 of the instrument attachment 30 through a spring-loaded hatch, or a threaded coupling, or any other mechanism, now known or later developed, that permits easy engagement and disengagement whilst protecting against leakage. In at least one alternate embodiment, the engagement end 36 of the instrument attachment 30 slots into the first end 52 of the imaging assembly 28. In at least one further embodiment, the curette device 22 provides an at least one quick-release button 55 for selectively disengaging the instrument attachment 30 from the imaging assembly 28.

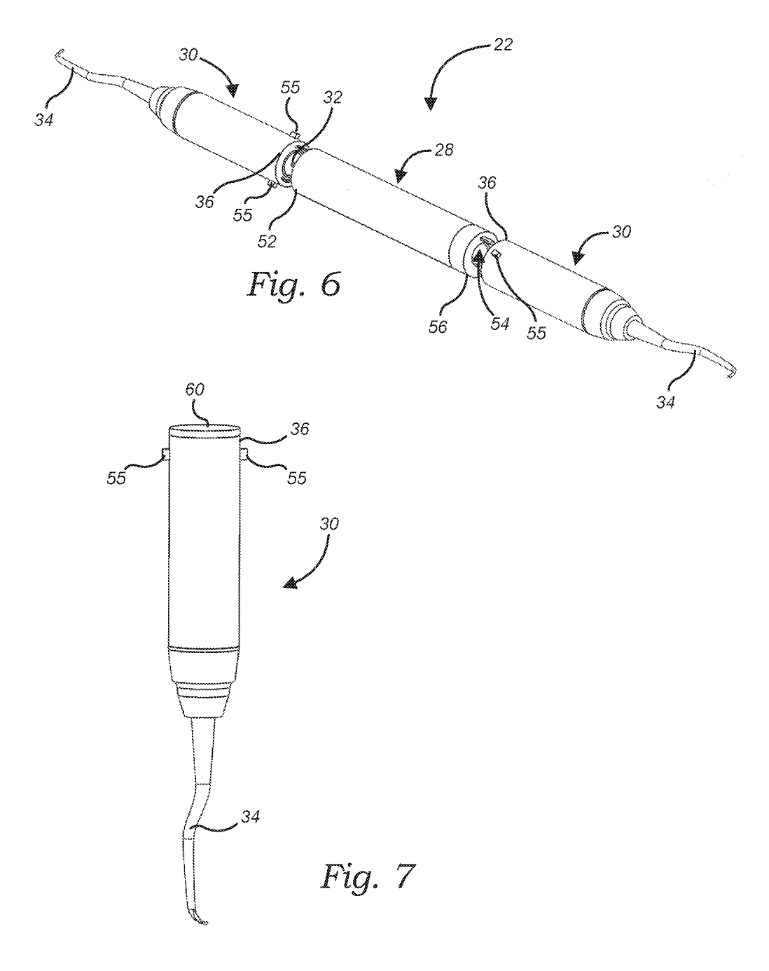

In at least one further embodiment, as illustrated in FIG. 6, an opposing second end 56 of the imaging assembly 28 is configured for being selectively engaged with the engagement end 36 of a further instrument attachment 30—thereby allowing the imaging assembly 28 to be simultaneously engaged with two different instrument attachments 30. In at least one such embodiment, the engagement end 36 of the further instrument attachment 30 and the second end 56 of the imaging assembly 28 cooperate to provide an optical port 54 therebetween, the optical port 54 configured for placing the at least one fiber optic bundle 32 of the further instrument attachment 30 in optical communication with each of the imaging sensor 48 and at least one light source 50, when the further instrument attachment 30 is removably engaged with the imaging assembly 28. In at least one embodiment, as illustrated in FIG. 4, the imaging assembly 28 provides a further imaging sensor 48 and a further at least one light source 50 configured for selectively interacting with the further instrument attachment 30. In at least one alternate embodiment, the imaging assembly 28 provides a single imaging sensor 48 and at least one light source 50 configured for selectively interacting with each of the two instrument attachments 30. In at least one embodiment, the imaging assembly 28 provides an at least one orientation sensor 58 (such as a gyrometer or accelerometer, for example), positioned and configured for determining the current orientation of the imaging assembly 28. Accordingly, in at least one such embodiment, where the imaging assembly 28 is engaged with two instrument attachments 30, the imaging assembly 28 is capable of determining which of the two instrument attachments 30 is in use at any given time (based on the orientation of the curette device 22) and so can automatically activate and deactivate the appropriate imaging sensor 48 and/or at least one light source 50.

In at least one embodiment, as illustrated in FIG. 7, the instrument attachment 30 provides an end cap 60 sized and configured for removable engagement with the engagement end 36 of the instrument attachment 30, thereby covering and protecting the at least one fiber optic bundle 32 when the instrument attachment 30 is not engaged with the imaging assembly 28.

As mentioned above, in at least one embodiment, the reflected light is captured by the at least one return fiber and transmitted along the return fiber to the imaging sensor 48 positioned within the imaging assembly 28. In at least one such embodiment, the reflected light is an analog image, which is then processed by the imaging sensor 48 to convert the reflected light into a digital image. In at least one embodiment, a fiber optic controller 62 is also positioned within the imaging assembly 28 and configured for controlling the imaging sensor 48 and the at least one light source 50 (where the at least one light source 50 is provided via the at least one light fiber). The resulting digital data from the fiber optic controller 62 is transmitted to an at least one microprocessor 64, also positioned within the imaging assembly 28, which in turn processes the data to be transmitted to the at least one computing device 24 and/or imaging display 26 via an at least one transceiver 66 positioned within the imaging assembly 28. The data (i.e., the images captured by the curette device 22, are then displayed in real-time via the at least one computing device 24 and/or imaging display 26 for the user to see. In at least one embodiment, the at least one transceiver 66 is configured for transmitting the data via a wireless communication protocol, such as WiFi, Bluetooth, NFC, or any other wireless communication protocol (or combination of such protocols) now known or later developed.

It should be noted that while, in at least one embodiment, the at least one image is provided to the user utilizing fiber optic technology, in at least one alternate embodiment, the at least one image is provided by a micro-miniaturized imaging system positioned within the instrument attachment 30, with the at least one image being transmitted wirelessly from the curette device 22 to the at least one computing device 24 or imaging display 26 in view of the user. In still further alternate embodiments, any other imaging technology, now known or later developed, capable of substantially carrying out the imaging functionality herein described, may be substituted. Thus, while the system 20 is discussed herein primarily in the context of fiber optics, the present invention should not be read as being so limited. In at least one embodiment, the imaging assembly 28 further provides a profilometer (not shown) positioned and configured for measuring a surface smoothness of the patient target area on which the curette device 22 is operating via the at least one fiber optic bundle 32.

In at least one embodiment, the curette device 22 is provides a button (not shown) in electrical communication with the microprocessor 64, which can be selectively pressed by the user in order to start and stop image capturing by the curette device 22. In at least one alternate embodiment, the curette device 22 is in communication with a foot pedal (not shown) which can be selectively pressed by the user in order to start and stop image capturing by the curette device 22. In at least one embodiment, the curette device 22 is configured for capturing images in a continuous manner—either as a series of still images or as a video. In at least one further embodiment, the curette device 22 is configured for allowing the user to capture a single image at a time with each manual press of the button or foot pedal. Other embodiments can utilize other means for the hygienist to use to signal the capture of an image. In at least one embodiment, images can also be saved as image or movie clip files or as a part of dental, oral or other charting procedures.

In at least one embodiment, the imaging assembly 28 provides an at least one battery 68—rechargeable and/or replaceable—configured for selectively powering the curette device 22. In at least one embodiment, where the at least one battery 68 is rechargeable, the battery 68 is capable of being recharged via inductive charging, or via any other contactless charging technology, now known or later developed. In at least one such embodiment, the wireless power transfer is accomplished using auto-tuning magnetically coupled resonators allowing for relaxed alignment requirements as the imaging assembly 28 is set down. This allows for the implementation of power transmission coils to be incorporated into an accompanying tool tray (i.e., a tray on which the user may keep a number of different tools, including the curette device 22) and the power transfer system auto-tuning to match the highest power transfer efficiency, resulting in a charge every time the curette device 22 is set down on the tray. In another embodiment, the curette device 22 utilizes inductive coupling to utilize a unique cradle to sit in allowing the imaging assembly 28 to be charged each and every time the user sets the curette device 22 down in an accompanying cradle. In still further embodiments, any other means for charging and powering the curette device 22, now known or later developed, may be substituted. In at least one embodiment, a power controller 70 provides battery 68 charging and battery 68 state control functions, and distributes regulated power to all electrical components in the curette device 22.

Furthermore, because all of the electrical components are tether-less and self-contained within the imaging assembly 28 in at least one embodiment, the curette device 22 is capable of being operated with a single hand, and by a single user. Additionally, given the removable engagement between the instrument attachment 30 and the imaging assembly 28, various types of instrument attachments 30 may be quickly and easily removably engaged with the imaging assembly 30 during use without having to reconnect any wires (or re-establish any wireless connections) to the at least one computing device 24 and/or imaging display 26. Furthermore, with all of the electrical components being positioned within the imaging assembly 28, the various instrument attachments 30 may be quickly and easily disengaged for regular instrument sterilization (as well as separate sterilization procedures for the imaging assembly 28 as needed). In at least one embodiment, the at least one optical port 54 is designed to be self-cleaning, either through specific deposit-repellent material, or mechanical design (or a combination of the two), or through any other technology now known or later developed.

In at least one embodiment, proposed uses of the system 20 include improved visualization of dental, oral as well as gingival and subgingival hard and soft tissues and structures, as well as dental restorations and implants for: (1) assessing the need for therapy, including but not limited to surgical or non-surgical periodontal procedures by identification of structures or pathologies or other conditions of interest such as bony defects and/or embedded deposits that may otherwise be left undetected; (2) identification of pathologies such as but not limited to oral, dental, mucosal, tooth and pocket pathologies; (3) directing real-time monitoring of scaling, root planning, surgical and other periodontal, oral, mucosal, dental and other treatment processes for the purposes of, but not limited to, determining progress and point of completion, thereby optimizing treatment, reducing procedure time and trauma on the tissues, increasing successful outcomes, and achieving other benefits; (4) permitting high-resolution visual assessment and monitoring of the efficacy of said and other treatment procedures; (5) assisting with diagnostic processes, including evaluation of tooth or implant health; and (6) evaluation of restorative interventions.

Thus, in at least one embodiment, the system 20 may be used clinically as an illumination and/or visualization tool, as well as a periodontal curette/scaler for diagnostic and evaluation purposes, along with therapeutic interventions such as removing etiological factors or pathologies from the tooth/root or pocket surfaces. Accordingly, in at least one such embodiment, the system 20 is capable of providing benefits such as: less diagnostic trauma as well as more thorough instrumentation; reduced instrumentation trauma to the soft tissues; and improved diagnosis of a wide range of conditions such as root pathologies, anatomical anomalies, faulty restorative margins or micro-leakage, residual cement, and peri-implantitis. In at least one embodiment, the system 20 allows direct assessment of patient's preventive and/or therapeutic needs including non-surgical and/or surgical periodontal therapy, detection of etiological factors and pathologies and other conditions, and determination of milestones such as an end point of therapy. Additionally, in at least one embodiment, the length of time required for diagnosis, therapy and other items may be reduced, thereby lessening fatigue and therapeutic costs and providing other benefits for patient and user.

As mentioned above, in further embodiments, the system 20 may be utilized in any other context—now known or later developed—where a curette or similar instrument might be utilized, such as removing impacted ear wax, curettage of the uterus, excision of adenoids, etc. Accordingly, the system 20 and associated methods described herein should not be read as being so limited. Instead, the system 20 and associated methods described herein are provided for illustrative purposes only in order to facilitate a more complete understanding of representative embodiments now contemplated. These examples are intended to be a mere subset of all possible contexts in which the system 20 may be utilized.

Aspects of the present specification may also be described as follows:

1. A wireless optical curette device for allowing a user to visually examine in real-time and debride a target surface within a cavity of a patient, the device comprising: an at least one instrument attachment providing an at least one fiber optic bundle extending between a working end of the instrument attachment and an opposing engagement end of the instrument attachment; an imaging assembly having a first end selectively engagable with the engagement end of the at least one instrument attachment, the imaging assembly providing: an at least one light source in selective optical communication with the at least one fiber optic bundle and configured for delivering an amount of controlled light to the target surface via the at least one fiber optic bundle; an at least one imaging sensor positioned within the imaging assembly in selective optical communication with the at least one fiber optic bundle and configured for receiving an amount of controlled light, via the at least one fiber optic bundle, reflected from the target surface, and converting the reflected light into an at least one digital image of the target surface; and an at least one microprocessor positioned within the imaging assembly and configured for wirelessly transmitting the at least one digital image, via an at least one transceiver positioned within the imaging assembly, to at least one of an at least one computing device and an at least one imaging display for displaying the at least one digital image in real-time; and the engagement end of the at least one instrument attachment and the first end of the imaging assembly cooperating to provide an optical port therebetween, the optical port configured for placing the at least one fiber optic bundle of the instrument attachment in optical communication with each of the at least one imaging sensor and at least one light source, when the instrument attachment is selectively engaged with the imaging assembly; whereby, the imaging assembly is capable of being removably engaged quickly and easily with a wide variety of different instrument attachments.

2. The wireless optical curette device according to embodiment 1, wherein the working end of the at least one instrument attachment provides a lens in optical communication with the at least one fiber optic bundle, the lens providing a field of view showing the target surface relative to the position of the working end of the instrument attachment.

3. The wireless optical curette device according to embodiments 1-2, wherein the lens is a trimmed and polished terminal end of the at least one fiber optic bundle.

4. The wireless optical curette device according to embodiments 1-3, wherein the field of view of the lens is in substantially the same plane as a face of the working end of the instrument attachment during use of the curette device.

5. The wireless optical curette device according to embodiments 1-4, wherein a focal length of the lens is set to allow for the lens to sit close to a cutting edge of the working end of the instrument attachment with relatively minimal impact on the overall width of the working end.

6. The wireless optical curette device according to embodiments 1-5, wherein the at least one light source is in selective optical communication with an at least one light fiber of the at least one fiber optic bundle.

7. The wireless optical curette device according to embodiments 1-6, wherein the at least one imaging sensor is in selective optical communication with an at least one return fiber of the at least one fiber optic bundle.

8. The wireless optical curette device according to embodiments 1-7, wherein the at least one light source is positioned within the imaging assembly.

9. The wireless optical curette device according to embodiments 1-8, wherein the at least one light source is an at least one LED.

10. The wireless optical curette device according to embodiments 1-9, wherein the at least one light source provides two or more different colors of controlled light for improving contrast of the at least one digital image.

11. The wireless optical curette device according to embodiments 1-10, wherein the imaging assembly provides a polarization filter for reducing specular reflection of the reflected light received by the at least one imaging sensor.

12. The wireless optical curette device according to embodiments 1-11, wherein the at least one light source is external to the imaging assembly.

13. The wireless optical curette device according to embodiments 1-12, wherein selective engagement between the at least one instrument attachment and the imaging assembly is achieved through the first end of the imaging assembly slotting into the engagement end of the instrument attachment through at least one of a spring-loaded hatch and a threaded coupling, thereby permitting easy engagement and disengagement whilst protecting against leakage.

14. The wireless optical curette device according to embodiments 1-13, wherein selective engagement between the at least one instrument attachment and the imaging assembly is achieved through the engagement end of the instrument attachment slotting into the first end of the imaging assembly through at least one of a spring-loaded hatch and a threaded coupling, thereby permitting easy engagement and disengagement whilst protecting against leakage.

15. The wireless optical curette device according to embodiments 1-14, wherein an opposing second end of the imaging assembly is configured for being selectively engaged with an engagement end of a further instrument attachment, thereby allowing the imaging assembly to be simultaneously engaged with two different instrument attachments.

16. The wireless optical curette device according to embodiments 1-15, wherein the engagement end of the further instrument attachment and the second end of the imaging assembly cooperate to provide an optical port therebetween, the optical port configured for placing an at least one fiber optic bundle of the further instrument attachment in optical communication with each of the at least one imaging sensor and at least one light source, when the further instrument attachment is selectively engaged with the imaging assembly.

17. The wireless optical curette device according to embodiments 1-16, wherein selective engagement between the further instrument attachment and the imaging assembly is achieved through the second end of the imaging assembly slotting into the engagement end of the further instrument attachment through at least one of a spring-loaded hatch and a threaded coupling, thereby permitting easy engagement and disengagement whilst protecting against leakage.

18. The wireless optical curette device according to embodiments 1-17, wherein selective engagement between the further instrument attachment and the imaging assembly is achieved through the engagement end of the further instrument attachment slotting into the second end of the imaging assembly through at least one of a spring-loaded hatch and a threaded coupling, thereby permitting easy engagement and disengagement whilst protecting against leakage.

19. The wireless optical curette device according to embodiments 1-18, wherein the imaging assembly provides an at least one orientation sensor positioned and configured for determining a current orientation of the imaging assembly and, in turn, which of the two instrument attachments is in use at any given time.

20. The wireless optical curette device according to embodiments 1-19, wherein the at least one instrument attachment provides an end cap sized and configured for removable engagement with the engagement end of the instrument attachment, thereby covering and protecting the at least one fiber optic bundle when the instrument attachment is not engaged with the imaging assembly.

21. The wireless optical curette device according to embodiments 1-20, wherein the imaging assembly provides an at least one fiber optic controller positioned and configured for controlling the at least one imaging sensor and at least one light source.

22. The wireless optical curette device according to embodiments 1-21, wherein the imaging assembly provides an at least one profilometer positioned and configured for measuring a surface smoothness of the target surface.

23. The wireless optical curette device according to embodiments 1-22, further comprising an at least one button in electrical communication with the at least one microprocessor, the button configured for being selectively pressed by the user in order to start and stop the capturing of digital images.

24. The wireless optical curette device according to embodiments 1-23, wherein the imaging assembly provides an at least one battery.

25. The wireless optical curette device according to embodiments 1-24, wherein the at least one battery is positioned and configured for being selectively recharged via inductive charging.

26. A wireless optical curette device for allowing a user to visually examine in real-time and debride a target surface within a cavity of a patient, the device comprising: a first instrument attachment providing an at least one fiber optic bundle extending between a working end of the first instrument attachment and an opposing engagement end of the first instrument attachment; a second instrument attachment providing an at least one fiber optic bundle extending between a working end of the second instrument attachment and an opposing engagement end of the second instrument attachment; an imaging assembly having a first end selectively engagable with the engagement end of the first instrument attachment, and a second end selectively engagable with the engagement end of the second instrument attachment, the imaging assembly providing: an at least one light source in selective optical communication with the at least one fiber optic bundle of the first and second instrument attachments and configured for delivering an amount of controlled light to the target surface via the at least one fiber optic bundle; an at least one imaging sensor positioned within the imaging assembly in selective optical communication with the at least one fiber optic bundle of the first and second instrument attachments and configured for receiving an amount of controlled light, via the at least one fiber optic bundle, reflected from the target surface, and converting the reflected light into an at least one digital image of the target surface; an at least one microprocessor positioned within the imaging assembly and configured for wirelessly transmitting the at least one digital image, via an at least one transceiver positioned within the imaging assembly, to at least one of an at least one computing device and an at least one imaging display for displaying the at least one digital image in real-time; and an at least one orientation sensor positioned within the imaging assembly and configured for determining a current orientation of the imaging assembly and, in turn, which of the first and second instrument attachments is in use at any given time; the engagement end of the first instrument attachment and the first end of the imaging assembly cooperating to provide a first optical port therebetween, the first optical port configured for placing the at least one fiber optic bundle of the first instrument attachment in optical communication with each of the at least one imaging sensor and at least one light source, when the first instrument attachment is selectively engaged with the imaging assembly; and the engagement end of the second instrument attachment and the second end of the imaging assembly cooperating to provide a second optical port therebetween, the second optical port configured for placing the at least one fiber optic bundle of the second instrument attachment in optical communication with each of the at least one imaging sensor and at least one light source, when the second instrument attachment is selectively engaged with the imaging assembly; whereby, each of the first and second ends of the imaging assembly is capable of being removably engaged quickly and easily with a wide variety of different instrument attachments.

27. A wireless optical curette system for allowing a user to visually examine in real-time and debride a target surface within a cavity of a patient, the system comprising: a wireless optical curette device in selective wireless communication with at least one of an at least one computing device and an at least one imaging display and configured for capturing and transmitting digital images of the target surface thereto in real-time, the curette device comprising: an at least one instrument attachment providing an at least one fiber optic bundle extending between a working end of the instrument attachment and an opposing engagement end of the instrument attachment; an imaging assembly having a first end selectively engagable with the engagement end of the at least one instrument attachment, the imaging assembly providing: an at least one light source in selective optical communication with the at least one fiber optic bundle and configured for delivering an amount of controlled light to the target surface via the at least one fiber optic bundle; an at least one imaging sensor positioned within the imaging assembly in selective optical communication with the at least one fiber optic bundle and configured for receiving an amount of controlled light, via the at least one fiber optic bundle, reflected from the target surface, and converting the reflected light into an at least one digital image of the target surface; and an at least one microprocessor positioned within the imaging assembly and configured for wirelessly transmitting the at least one digital image, via an at least one transceiver positioned within the imaging assembly, to at least one of the at least one computing device and at least one imaging display for displaying the at least one digital image in real-time; and the engagement end of the at least one instrument attachment and the first end of the imaging assembly cooperating to provide an optical port therebetween, the optical port configured for placing the at least one fiber optic bundle of the instrument attachment in optical communication with each of the at least one imaging sensor and at least one light source, when the instrument attachment is selectively engaged with the imaging assembly; whereby, the imaging assembly is capable of being removably engaged quickly and easily with a wide variety of different instrument attachments.

28. The system according to embodiment 27, wherein the at least one imaging display is configured for receiving and displaying the at least one digital image transmitted by the curette device.

29. The system according to embodiments 27-28, wherein the at least one computing device is configured for receiving and displaying the at least one digital image transmitted by the curette device.

30. The system according to embodiments 27-29, wherein the at least one computing device is further configured for storing the at least one digital image transmitted by the curette device.

31. The system according to embodiments 27-30, wherein the working end of the instrument attachment provides a lens in optical communication with the at least one fiber optic bundle, the lens providing a field of view showing the target surface relative to the position of the working end of the instrument attachment.

32. The system according to embodiments 27-31, wherein the lens is a trimmed and polished terminal end of the at least one fiber optic bundle.

33. The system according to embodiments 27-32, wherein the field of view of the lens is in substantially the same plane as a face of the working end of the instrument attachment during use of the curette device.

34. The system according to embodiments 27-33, wherein a focal length of the lens is set to allow for the lens to sit close to a cutting edge of the working end of the instrument attachment with relatively minimal impact on the overall width of the working end.

35. The system according to embodiments 27-34, wherein the at least one light source is in selective optical communication with an at least one light fiber of the at least one fiber optic bundle.

36. The system according to embodiments 27-35, wherein the at least one imaging sensor is in selective optical communication with an at least one return fiber of the at least one fiber optic bundle.

37. The system according to embodiments 27-36, wherein the at least one light source is positioned within the imaging assembly.

38. The system according to embodiments 27-37, wherein the at least one light source is an at least one LED.

39. The system according to embodiments 27-38, wherein the at least one light source provides two or more different colors of controlled light for improving contrast of the at least one digital image.

40. The system according to embodiments 27-39, wherein the imaging assembly provides a polarization filter for reducing specular reflection of the reflected light received by the at least one imaging sensor.

41. The system according to embodiments 27-40, wherein the at least one light source is external to the imaging assembly.

42. The system according to embodiments 27-41, wherein selective engagement between the at least one instrument attachment and the imaging assembly is achieved through the first end of the imaging assembly slotting into the engagement end of the instrument attachment through at least one of a spring-loaded hatch and a threaded coupling, thereby permitting easy engagement and disengagement whilst protecting against leakage.

43. The system according to embodiments 27-42, wherein selective engagement between the at least one instrument attachment and the imaging assembly is achieved through the engagement end of the instrument attachment slotting into the first end of the imaging assembly through at least one of a spring-loaded hatch and a threaded coupling, thereby permitting easy engagement and disengagement whilst protecting against leakage.

44. The system according to embodiments 27-43, wherein an opposing second end of the imaging assembly is configured for being selectively engaged with an engagement end of a further instrument attachment, thereby allowing the imaging assembly to be simultaneously engaged with two different instrument attachments.

45. The system according to embodiments 27-44, wherein the engagement end of the further instrument attachment and the second end of the imaging assembly cooperate to provide an optical port therebetween, the optical port configured for placing an at least one fiber optic bundle of the further instrument attachment in optical communication with each of the at least one imaging sensor and at least one light source, when the further instrument attachment is selectively engaged with the imaging assembly.

46. The system according to embodiments 27-45, wherein selective engagement between the further instrument attachment and the imaging assembly is achieved through the second end of the imaging assembly slotting into the engagement end of the further instrument attachment through at least one of a spring-loaded hatch and a threaded coupling, thereby permitting easy engagement and disengagement whilst protecting against leakage.

47. The system according to embodiments 27-46, wherein selective engagement between the further instrument attachment and the imaging assembly is achieved through the engagement end of the further instrument attachment slotting into the second end of the imaging assembly through at least one of a spring-loaded hatch and a threaded coupling, thereby permitting easy engagement and disengagement whilst protecting against leakage.

48. The system according to embodiments 27-47, wherein the imaging assembly provides an at least one orientation sensor positioned and configured for determining a current orientation of the imaging assembly and, in turn, which of the two instrument attachments is in use at any given time.

49. The system according to embodiments 27-48, wherein the at least one instrument attachment provides an end cap sized and configured for removable engagement with the engagement end of the instrument attachment, thereby covering and protecting the at least one fiber optic bundle when the instrument attachment is not engaged with the imaging assembly.

50. The system according to embodiments 27-49, wherein the imaging assembly provides an at least one fiber optic controller positioned and configured for controlling the at least one imaging sensor and at least one light source.

51. The system according to embodiments 27-50, wherein the imaging assembly provides an at least one profilometer positioned and configured for measuring a surface smoothness of the target surface.

52. The system according to embodiments 27-51, further comprising an at least one button in electrical communication with the at least one microprocessor, the button configured for being selectively pressed by the user in order to start and stop the capturing of digital images.

53. The system according to embodiments 27-52, wherein the imaging assembly provides an at least one battery.

54. The system according to embodiments 27-53, wherein the at least one battery is positioned and configured for being selectively recharged via inductive charging.

In closing, regarding the exemplary embodiments of the present invention as shown and described herein, it will be appreciated that a wireless optical curette system is disclosed and configured for allowing a user of the system to visually examine in real-time and debride a cavity of a patient. Because the principles of the invention may be practiced in a number of configurations beyond those shown and described, it is to be understood that the invention is not in any way limited by the exemplary embodiments, but is generally directed to a wireless optical curette system and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

It should be understood that the logic code, programs, modules, processes, methods, and the order in which the respective elements of each method are performed are purely exemplary. Depending on the implementation, they may be performed in any order or in parallel, unless indicated otherwise in the present disclosure. Further, the logic code is not related, or limited to any particular programming language, and may comprise one or more modules that execute on one or more processors in a distributed, non-distributed, or multiprocessing environment.

The methods as described above may be used in the fabrication of integrated circuit chips. The resulting integrated circuit chips can be distributed by the fabricator in raw wafer form (that is, as a single wafer that has multiple unpackaged chips), as a bare die, or in a packaged form. In the latter case, the chip is mounted in a single chip package (such as a plastic carrier, with leads that are affixed to a motherboard or other higher level carrier) or in a multi-chip package (such as a ceramic carrier that has either or both surface interconnections or buried interconnections). In any case, the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A wireless optical curette device for allowing a user to visually examine in real-time and debride a target surface within a cavity of a patient, the device comprising:
an at least one instrument attachment providing an at least one fiber optic bundle extending between a working end of the instrument attachment and an opposing engagement end of the instrument attachment;
an imaging assembly having a first end selectively engagable with the engagement end of the at least one instrument attachment, the imaging assembly providing:
an at least one light source in selective optical communication with the at least one fiber optic bundle and configured for delivering an amount of controlled light to the target surface via the at least one fiber optic bundle;
an at least one imaging sensor positioned within the imaging assembly in selective optical communication with the at least one fiber optic bundle and configured for receiving an amount of controlled light, via the at least one fiber optic bundle, reflected from the target surface, and converting the reflected light into an at least one digital image of the target surface; and
an at least one microprocessor positioned within the imaging assembly and configured for wirelessly transmitting the at least one digital image, via an at least one transceiver positioned within the imaging assembly, to at least one of an at least one computing device and an at least one imaging display for displaying the at least one digital image in real-time; and
the engagement end of the at least one instrument attachment and the first end of the imaging assembly cooperating to provide an optical port therebetween, the optical port configured for placing the at least one fiber optic bundle of the instrument attachment in optical communication with each of the at least one imaging sensor and at least one light source, when the instrument attachment is selectively engaged with the imaging assembly;
whereby, the imaging assembly is capable of being removably engaged quickly and easily with a wide variety of different instrument attachments.

2. The wireless optical curette device of claim 1, wherein the working end of the at least one instrument attachment provides a lens in optical communication with the at least one fiber optic bundle, the lens providing a field of view showing the target surface relative to the position of the working end of the instrument attachment.

3. The wireless optical curette device of claim 2, wherein the lens is a trimmed and polished terminal end of the at least one fiber optic bundle.

4. The wireless optical curette device of claim 2, wherein the field of view of the lens is in substantially the same plane as a face of the working end of the instrument attachment during use of the curette device.

5. The wireless optical curette device of claim 2, wherein a focal length of the lens is set to allow for the lens to sit close to a cutting edge of the working end of the instrument attachment with relatively minimal impact on the overall width of the working end.

6. The wireless optical curette device of claim 1, wherein the at least one light source is in selective optical communication with an at least one light fiber of the at least one fiber optic bundle.

7. The wireless optical curette device of claim 1, wherein the at least one imaging sensor is in selective optical communication with an at least one return fiber of the at least one fiber optic bundle.

8. The wireless optical curette device of claim 1, wherein the at least one light source is positioned within the imaging assembly.

9. The wireless optical curette device of claim 8, wherein the at least one light source is an at least one LED.

10. The wireless optical curette device of claim 9, wherein the at least one light source provides two or more different colors of controlled light for improving contrast of the at least one digital image.

11. The wireless optical curette device of claim 8, wherein the imaging assembly provides a polarization filter for reducing specular reflection of the reflected light received by the at least one imaging sensor.

12. The wireless optical curette device of claim 1, wherein selective engagement between the at least one instrument attachment and the imaging assembly is achieved through the first end of the imaging assembly slotting into the engagement end of the instrument attachment through at least one of a spring-loaded hatch and a threaded coupling, thereby permitting easy engagement and disengagement whilst protecting against leakage.

13. The wireless optical curette device of claim 1, wherein an opposing second end of the imaging assembly is configured for being selectively engaged with an engagement end of a further instrument attachment, thereby allowing the imaging assembly to be simultaneously engaged with two different instrument attachments.

14. The wireless optical curette device of claim 13, wherein the engagement end of the further instrument attachment and the second end of the imaging assembly cooperate to provide an optical port therebetween, the optical port configured for placing an at least one fiber optic bundle of the further instrument attachment in optical communication with each of the at least one imaging sensor and at least one light source, when the further instrument attachment is selectively engaged with the imaging assembly.

15. The wireless optical curette device of claim 14, wherein selective engagement between the further instrument attachment and the imaging assembly is achieved through the second end of the imaging assembly slotting into the engagement end of the further instrument attachment through at least one of a spring-loaded hatch and a threaded coupling, thereby permitting easy engagement and disengagement whilst protecting against leakage.

16. The wireless optical curette device of claim 13, wherein the imaging assembly provides an at least one orientation sensor positioned and configured for determining a current orientation of the imaging assembly and, in turn, which of the two instrument attachments is in use at any given time.

17. The wireless optical curette device of claim 1, wherein the at least one instrument attachment provides an end cap sized and configured for removable engagement with the engagement end of the instrument attachment, thereby covering and protecting the at least one fiber optic bundle when the instrument attachment is not engaged with the imaging assembly.

18. The wireless optical curette device of claim 1, wherein the imaging assembly provides an at least one profilometer positioned and configured for measuring a surface smoothness of the target surface.

19. A wireless optical curette device for allowing a user to visually examine in real-time and debride a target surface within a cavity of a patient, the device comprising:
a first instrument attachment providing an at least one fiber optic bundle extending between a working end of the first instrument attachment and an opposing engagement end of the first instrument attachment;
a second instrument attachment providing an at least one fiber optic bundle extending between a working end of the second instrument attachment and an opposing engagement end of the second instrument attachment;
an imaging assembly having a first end selectively engagable with the engagement end of the first instrument attachment, and a second end selectively engagable with the engagement end of the second instrument attachment, the imaging assembly providing:
an at least one light source in selective optical communication with the at least one fiber optic bundle of the first and second instrument attachments and configured for delivering an amount of controlled light to the target surface via the at least one fiber optic bundle;
an at least one imaging sensor positioned within the imaging assembly in selective optical communication with the at least one fiber optic bundle of the first and second instrument attachments and configured for receiving an amount of controlled light, via the at least one fiber optic bundle, reflected from the target surface, and converting the reflected light into an at least one digital image of the target surface;
an at least one microprocessor positioned within the imaging assembly and configured for wirelessly transmitting the at least one digital image, via an at least one transceiver positioned within the imaging assembly, to at least one of an at least one computing device and an at least one imaging display for displaying the at least one digital image in real-time; and
an at least one orientation sensor positioned within the imaging assembly and configured for determining a current orientation of the imaging assembly and, in turn, which of the first and second instrument attachments is in use at any given time;
the engagement end of the first instrument attachment and the first end of the imaging assembly cooperating to provide a first optical port therebetween, the first optical port configured for placing the at least one fiber optic bundle of the first instrument attachment in optical communication with each of the at least one imaging sensor and at least one light source, when the first instrument attachment is selectively engaged with the imaging assembly; and
the engagement end of the second instrument attachment and the second end of the imaging assembly cooperating to provide a second optical port therebetween, the second optical port configured for placing the at least one fiber optic bundle of the second instrument attachment in optical communication with each of the at least one imaging sensor and at least one light source, when the second instrument attachment is selectively engaged with the imaging assembly;
whereby, each of the first and second ends of the imaging assembly is capable of being removably engaged quickly and easily with a wide variety of different instrument attachments.

20. A wireless optical curette system for allowing a user to visually examine in real-time and debride a target surface within a cavity of a patient, the system comprising:
a wireless optical curette device in selective wireless communication with at least one of an at least one computing device and an at least one imaging display and configured for capturing and transmitting digital images of the target surface thereto in real-time, the curette device comprising:
an at least one instrument attachment providing an at least one fiber optic bundle extending between a working end of the instrument attachment and an opposing engagement end of the instrument attachment;
an imaging assembly having a first end selectively engagable with the engagement end of the at least one instrument attachment, the imaging assembly providing:
an at least one light source in selective optical communication with the at least one fiber optic bundle and configured for delivering an amount of controlled light to the target surface via the at least one fiber optic bundle;
an at least one imaging sensor positioned within the imaging assembly in selective optical communication with the at least one fiber optic bundle and configured for receiving an amount of controlled light, via the at least one fiber optic bundle, reflected from the target surface, and converting the reflected light into an at least one digital image of the target surface; and
an at least one microprocessor positioned within the imaging assembly and configured for wirelessly transmitting the at least one digital image, via an at least one transceiver positioned within the imaging assembly, to at least one of the at least one computing device and at least one imaging display for displaying the at least one digital image in real-time; and
the engagement end of the at least one instrument attachment and the first end of the imaging assembly cooperating to provide an optical port therebetween, the optical port configured for placing the at least one fiber optic bundle of the instrument attachment in optical communication with each of the at least one imaging sensor and at least one light source, when the instrument attachment is selectively engaged with the imaging assembly;
whereby, the imaging assembly is capable of being removably engaged quickly and easily with a wide variety of different instrument attachments.
